(19) United States
(12) Patent Application Publication
(10) Pub. No.: US 2018/0153526 A1
(43) Pub. Date: Jun. 7, 2018

(54) FUNCTIONAL COVER FOR BIOPSY DEVICE

(71) Applicant: Devicor Medical Products, Inc., Cincinnati, OH (US)

(72) Inventors: Andrew Paul Nock, Cincinnati, OH (US); Douglas Hartman, Batesville, IN (US); Melody L. Stamper, Batavia, OH (US); Margaret Meta Brumm, Marquette, MI (US)

(21) Appl. No.: 15/829,464

(22) Filed: Dec. 1, 2017

Related U.S. Application Data
(60) Provisional application No. 62/429,356, filed on Dec. 2, 2016.

(51) Int. Cl. A61B 10/02

(52) U.S. Cl. CPC A61B 10/0266

(57) ABSTRACT

A biopsy device includes a reusable body, a disposable probe, and a sterile cover. The reusable body contains a drive mechanism. The disposable probe is adapted to couple the drive mechanism of the reusable body in use. The disposable probe has a body and a needle extending distally from the body. The needle is movably coupled to the drive mechanism. The reusable body has at least one button for operating the probe. The sterile cover is shaped and configured to couple to the reusable body. The sterile cover includes a contact area for access to the at least one button.

### PRIORITY

The present application claims priority to U.S. Provisional Patent Application No. 62/429,356, entitled “Functional Cover for Biopsy Device,” filed on Dec. 2, 2016, the disclosure of which is hereby incorporated by referenced herein in its entirety.

### BACKGROUND

A biopsy is the removal of a tissue sample from a patient to enable examination of the tissue for signs of cancer or other disorders. Tissue samples may be obtained in a variety of ways using various medical procedures involving a variety of the sample collection devices. For example, biopsies may be open procedures (surgically removing tissue after creating an incision) or percutaneous procedures (e.g. by fine needle aspiration, core needle biopsy, or vacuum assisted biopsy). After the tissue sample is collected, the tissue sample may be analyzed at a lab (e.g. a pathology lab, biomedical lab, etc.) that is set up to perform the appropriate tests (such as histological).

Merely exemplary biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 5,928,164, entitled “Apparatus for Automated Biopsy and Collection of Soft Tissue,” issued Jul. 27, 1999; U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,118,755, entitled “Biopsy Sample Storage,” issued Feb. 21, 2012; U.S. Pat. No. 8,206,316, entitled “Tetherless Biopsy Device with Reusable Portion,” issued Jun. 26, 2012; U.S. Pat. No. 8,241,226, entitled “Biopsy Device with Rotatable Tissue Sample Holder,” issued Aug. 14, 2012; U.S. Pat. No. 8,764,680, entitled “Handheld Biopsy Device with Needle Firing,” issued Jul. 1, 2014; U.S. Pat. No. 8,801,742, entitled “Needle Assembly and Blade Assembly for Biopsy Device,” issued Aug. 12, 2014; U.S. Pat. No. 8,938,285, entitled “Access Chamber and Markers for Biopsy Device,” issued Jan. 20, 2015; U.S. Pat. No. 8,858,465, entitled “Biopsy Device with Motorized Needle Firing,” issued Oct. 14, 2014; U.S. Pat. No. 9,326,755, entitled “Biopsy Device Tissue Sample Holder with Bulk Chamber and Pathology Chamber,” issued May 3, 2016; and U.S. Pat. No. 9,345,457, entitled “Presentation of Biopsy Sample by Biopsy Device,” issued May 24, 2016. The disclosure of each of the above-cited U.S. patents is incorporated by reference herein.

Additional exemplary biopsy devices and biopsy system components are disclosed in U.S. Pat. Pub. No. 2006/0074345, entitled “Biopsy Apparatus and Method,” published Apr. 6, 2006, now abandoned; U.S. Pat. Pub. No. 2009/0131821, entitled “Graphical User Interface For Biopsy System Control Module,” published May 21, 2009, now abandoned; U.S. Pat. Pub. No. 2010/0160819, entitled “Biopsy Device with Central Thumbwheel,” published Jun. 24, 2010; and U.S. Pat. Pub. No. 2013/0324882, entitled “Control for Biopsy Device,” published Dec. 5, 2013, now abandoned. The disclosure of each of the above-cited U.S. patent application Publications, U.S. Non-Provisional patent applications, and U.S. Provisional patent applications is incorporated by reference herein.

While several systems and methods have been made and used for obtaining and processing a biopsy sample, it is believed that no one prior to the inventor has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary biopsy device including an exemplary holster coupled with an exemplary probe;

FIG. 2 depicts a perspective view of the biopsy device of FIG. 1, with an exemplary functional cover, which is a sterile cover coupled with the holster and the probe;

FIG. 3 depicts an exploded view of the biopsy device of FIG. 1, with the holster and functional cover decoupled from the probe;

FIG. 4 depicts a perspective view of the sterile cover of FIG. 2;

FIG. 5 depicts a front cross-sectional view of the sterile cover of FIG. 2, with the cross-section taken along line 5-5 of FIG. 4;

FIG. 6 depicts a rear cross-sectional view of the sterile cover of FIG. 2, with the cross-section taken along line 6-6 of FIG. 4; and

FIG. 7 depicts a perspective view of the holster coupled on the probe of FIG. 2, with the exemplary sterile cover removed.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

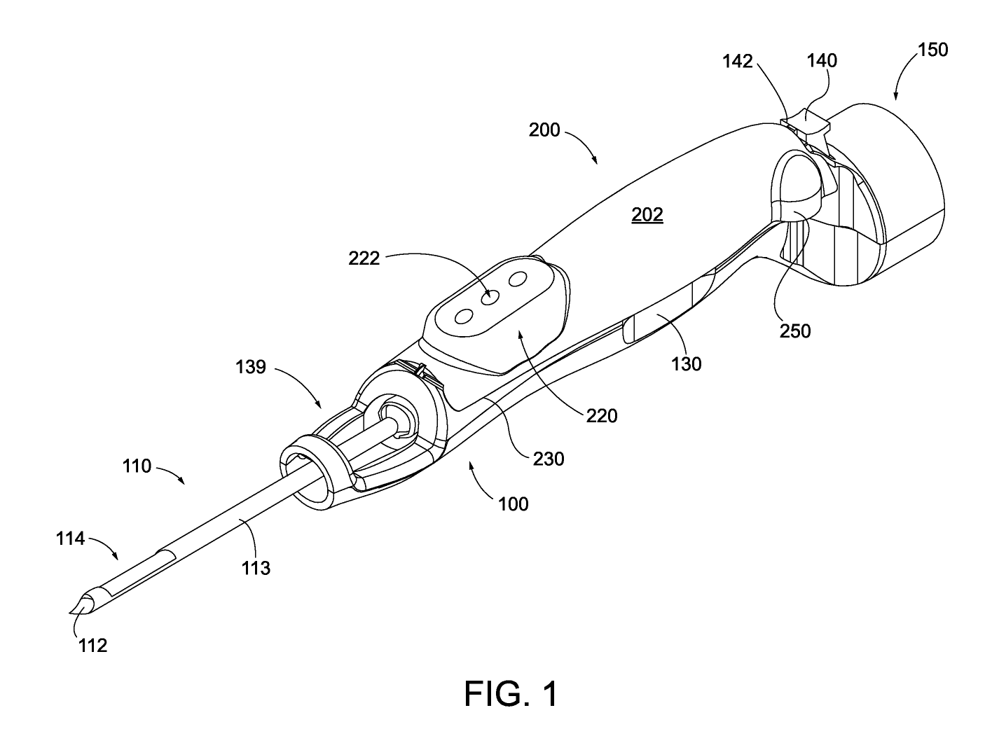

FIG. 1 shows an exemplary biopsy device (10) that may be used in a breast biopsy system. Biopsy device (10) of the present examples comprises a probe (100) and a holster (200). A needle (110) extends distally from probe (100) and is inserted into a patient's tissue to obtain tissue samples. These tissue samples are deposited in a tissue sample holder (150) at the proximal end of probe (100).

Holster (200) of the present example is selectively attachable to probe (100) to provide actuation of various components within probe (100). In the present configuration, holster (200) is a reusable component, while probe (100) and tissue sample holder (150) are disposable. It should be understood that the use of the term “holster” herein should not be read as requiring any portion of probe (100) to be inserted into any portion of holster (200). For instance, in the present example, holster (200) includes a set of prongs (not shown) or other retention features that are received by probe (100) to releasably secure probe (100) to holster (200). Probe (100) also includes a set of resilient tabs (not shown) or other suitable release features that may be pressed inwardly to disengage the prongs, such that an operator may simultaneously depress both of the tabs then pull probe (100) rearwardly and away from holster (200) to decouple probe (100) from holster (200). Of course, a variety of other types of structures, components, features, etc. (e.g., bayonet mounts, latches, clamps, clips, snap fittings, etc.) may be used to provide removable coupling of probe (100) and holster (200). Furthermore, in some biopsy devices (10), probe (100) and holster (200) may be of unitary or integral construction, such that the two components cannot be separated. By way of example only, in versions where probe (100) and holster (200) are provided as separable components, probe (100) may be provided as a disposable component, while holster (200) may be provided as a reusable component. Still other suitable structural and functional relationships between probe (100) and holster (200) will be apparent to those of ordinary skill in the art in view of the teachings herein.

Some variations of biopsy device (10) may include one or more sensors (not shown), in probe (100) and/or in holster (200), that is/are configured to detect when probe (100) is coupled with holster (200). Such sensors or other features may further be configured to permit only certain types of probes (100) and holsters (200) to be coupled together. In addition or in the alternative, such sensors may be configured to disable one or more functions of probes (100) and/or holsters (200) until a suitable probe (100) and holster (200) are coupled together. In one merely illustrative example, probe (100) includes a magnet (not shown) that is detected by a Hall Effect sensor (not shown) or some other type of sensor in holster (200) when probe (100) is coupled with holster (200). As yet another merely illustrative example, coupling of probe (100) with holster (200) may be detected using physical contact between conductive surfaces or electrodes, using RFID technology, and/or in numerous other ways as will be apparent to those of ordinary skill in the art in view of the teachings herein. Of course, such sensors and features may be varied or omitted as desired.

Biopsy device (10) of the present example is configured for handheld use, and be used under ultrasonic guidance. Of course, biopsy device (10) may instead be used under stereotactic guidance, MRI guidance, PEM guidance, BSGI guidance, or otherwise. It should also be understood that biopsy device (10) may be sized and configured such that biopsy device (10) may be operated by a single hand of an operator. In particular, an operator may grasp biopsy device (10), insert needle (110) into a patient's breast, and collect one or a plurality of tissue samples from within the patient's breast, all with just using a single hand. Such tissue samples may be deposited in tissue sample holder (113), and later retrieved from tissue sample holder (113) for analysis. While examples described herein often refer to the acquisition of biopsy samples from a patient's breast, it should be understood that biopsy device (100) may be used in a variety of other procedures for a variety of other purposes and in a variety of other parts of a patient's anatomy (e.g., prostate, thyroid, etc.). Various exemplary components, features, configurations, and operabilities of biopsy device (100) will be described in greater detail below; while other suitable components, features, configurations, and operabilities will be apparent to those of ordinary skill in the art in view of the teachings herein. Alternatively, an operator may grasp biopsy device (10) with more than one hand and/or with any desired assistance. In still other examples, biopsy device (10) can be configured to be secured to a table or other fixture without handheld operation.

In some settings, whether biopsy device (10) is handheld or mounted to a fixture, the operator may capture a plurality of tissue samples with just a single insertion of needle (110) into the patient's breast. Such tissue samples may be deposited in tissue sample holder (150), and later retrieved from tissue sample holder (150) for analysis. While examples described herein often refer to the acquisition of biopsy samples from a patient's breast, it should be understood that biopsy device (10) may be used in a variety of other procedures for a variety of other purposes and in a variety of other parts of a patient's anatomy (e.g., prostate, thyroid, etc.). Various exemplary components, features, configurations, and operabilities of biopsy device (10) will be described in greater detail below; while other suitable components, features, configurations, and operabilities will be apparent to those of ordinary skill in the art in view of the teachings herein.

Holster (200) of the present example includes an outer housing (202) that is configured to at least partially encompass the internal components of holster (200). Although not shown, it should be understood that holster (200) of the present example includes one or more motors and/or other actuators that are configured to drive various components of probe. To communicate power or movement to probe (100), holster (200) can include one or more gears. For instance, in some examples, one or more gears at least partially extend through an opening in outer housing (202). The opening in outer housing (202) can be configured to align with a corresponding opening associated with probe (100) to thereby permit the one or more gears of holster (200) to mesh with one or more corresponding gears of probe (100).

Although not shown, it should be understood that holster (200) may also include various cables that are configured to couple holster (200) to a control module or another control feature. Suitable cables may include electrical cables, rotary drive cables, pneumatic cables, or some combination thereof. Accordingly, it should be understood that in some examples, internal components within holster (200) may be powered by electrical power (electrical cables), rotary power (rotary drive cable), and/or pneumatic power (pneumatic cables). Alternatively, in some examples the cables are omitted entirely and holster (200) can be battery powered with motors and vacuum pumps being entirely contained within holster (200).

In some instances, it may be desirable for biopsy device (10) to include certain onboard controls to initiate operation of biopsy device (10). For instance, in the present example holster (200) includes button panel (220) with a plurality of individual buttons (222). Each individual button (222) can be used to communicate with a control module located either within holster (200) or externally from biopsy device (10). Through this communication various control parameters may be assigned to each button (222) such that an operator can manipulate the operation of biopsy device (10) via buttons (222). Although the present example is shown as including three buttons (222), in other examples any suitable number of buttons (222) may be used. In addition, or in the alternative, each button (222) may also be incorporated into holster (200) with indicators such as LED bulbs, a series of LED bulbs, and/or various other graphical indicators.

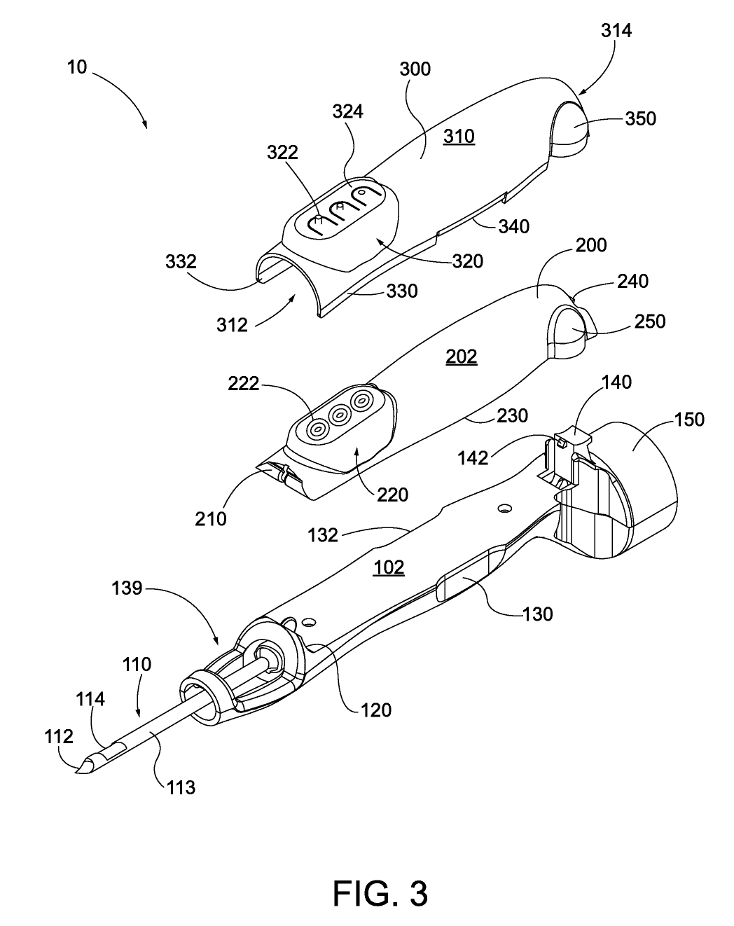

As described above with respect to holster (200), probe (100) is selectively couplable to holster (200) so that holster (200) may provide power or otherwise actuate probe (100). As can be best seen in FIG. 3, holster (200) includes a locating feature (210) at the distal end of holster (200). Holster (200) further includes a catch (240) at the proximal end of holster (200). Locating feature (210) is sized and configured to securely fit holster (200) into a corresponding receiving feature (120) of probe (100) when holster (200) is attached to probe (100) along outer housing (102). Catch (240) is sized and configured to securely attach holster (200) onto probe (100) by latching against a prong (142) of probe (100). Prong (142) extends distally from a latch (140) at the proximal end of probe (100). Latch (140) is operable to disengage prong (142) of probe (100) from catch (240) of holster (200) to allow holster (200) to be decoupled from outer housing (102) of probe (100). Of course, a variety of other types of structures, components, features, etc. (e.g., bayonet mounts, clamps, clips, snap fittings, etc.) may be used to provide removable coupling of probe (100) and holster (200). While only a few exemplary attachment configurations have been described for holster (200), other various configurations may be provided as will be apparent to those of ordinary skill in the art in view of the teachings herein. Furthermore, in some biopsy devices (10), probe (100) and holster (200) may be of unitary or integral construction, such that the two components cannot be separated. By way of example only, in versions where probe (100) and holster (200) are provided as separable components, probe (100) may be provided as a disposable component, while holster (200) may be provided as a reusable component. Still other suitable structural and functional relationships between probe (100) and holster (200) will be apparent to those of ordinary skill in the art in view of the teachings herein.

Probe (100) of the present example further includes a needle (110) extending distally from probe (100) that is inserted into a patient's tissue to obtain tissue samples. These tissue samples are deposited in a tissue sample holder (150) at the proximal end of probe (100). In some examples, a vacuum control module (not shown) is coupled with probe (100) via a valve assembly (not shown) and tubes (not shown), which is operable to selectively provide vacuum, saline, atmospheric air, and venting to probe (100). By way of example only, the internal components of the valve assembly may be configured and arranged as described in U.S. Pat. Pub. No. 2013/0218047, entitled “Biopsy Device Valve Assembly,” published Aug. 22, 2013, the disclosure of which is incorporated by reference herein.

Needle (110) of the present example comprises a cannula (113) having a piercing tip (112), and a lateral aperture (114) located proximal to tip (112). Tissue piercing tip (112) is configured to pierce and penetrate tissue, without requiring a high amount of force, and without requiring an opening to be pre-formed in the tissue prior to insertion of tip (112). Alternatively, tip (112) may be blunt (e.g., rounded, flat, etc.) if desired. By way of example only, tip (112) may be configured in accordance with any of the teachings in U.S. Pat. No. 8,801,742, entitled “Needle Assembly and Blade Assembly for Biopsy Device,” filed Jun. 1, 2011, the disclosure of which is incorporated by reference herein. As another merely illustrative example, tip (112) may be configured in accordance with at least some of the teachings in U.S. Pat. Pub. No. 2013/0144188, entitled “Biopsy Device with Slide-In Probe,” published Jun. 6, 2013, will issue on Nov. 8, 2016 as U.S. Pat. No. 9,486,186, the disclosure of which is incorporated by reference herein. Other suitable configurations that may be used for tip (112) will be apparent to those of ordinary skill in the art in view of the teachings herein.

Lateral aperture (114) is sized to receive prolapsed tissue during operation of device (10). A hollow tubular cutter (not shown) having a sharp distal edge (not shown) is located within needle (110). Cutter is operable to rotate and translate relative to needle (110) and past lateral aperture (114) to sever a tissue sample from tissue protruding through lateral aperture (114). For instance, cutter may be moved from an extended position to a retracted position, thereby “opening” lateral aperture (114) to allow tissue to protrude therethrough; then from the retracted position back to the extended position to sever the protruding tissue.

In some examples it may be desirable to rotate needle (110) to orient lateral aperture (114) at a plurality of desired angular positions about the longitudinal axis of needle (110). In the present example, needle (110) can be rotated by a motor disposed in probe (100) or holster (200). In other examples, needle (110) is manually rotatable by a thumbwheel on probe (100) or needle hub directly overmolded onto needle (110). Regardless, it should also be understood that, as with other components described herein, needle (110) may be varied, modified, substituted, or supplemented in a variety of ways; and that needle (110) may have a variety of alternative features, components, configurations, and functionalities. For instance, needle (110) may be constructed and operable in accordance with at least some of the teachings of U.S. Pat. No. 9,345,457, issued May 24, 2016, the disclosure of which is incorporated by reference herein; and/or in accordance with the teachings of any other reference cited herein.

As best seen in FIG. 3, outer housing (102) of probe (100) defines a pair of recesses (130, 132) on opposing lateral sides of probe (100). Recesses (130, 132) extend laterally and inwardly along the length of probe (100). In the present example, recesses (130, 132) are incorporated into probe (100) to provide access to at least a portion of holster (200). As will be described in greater detail below, this additional access to holster (200) permits selective attachment of a removable functional cover (300) to holster (200) to promote the sterility of holster (200) during a biopsy procedure. In some examples, recesses (130, 132) can also be included to enhance the grip of probe (100) during a biopsy procedure. However, in other examples any enhancement to grip may be entirely incidental, and recesses (130, 132) may only be added to promote access to holster (200).

Tissue sample holder (150) is selectively coupleable to the proximal end of probe (100). In some examples, tissue sample holder (150) may be configured to operate in two discrete sample collection modes—a bulk tissue collection mode and an individual tissue collection moved. By way of example only, tissue sample holder (150) may be constructed and operable in accordance with at least some of the teachings of U.S. application No. [Atty. Docket No. LEI 20007-SO-US.0642286], entitled “Multi-Chamber Tissue Sample Cup for Biopsy Device,” filed on an even date herewith, the disclosure of which is incorporated by reference herein. By way of further example, tissue sample holder (150) may be constructed and operable in accordance with at least some of the teachings of any of the other references cited herein; and/or in any other suitable fashion.

The distal portion of probe (100) further includes a tissue sample window (139) disposed proximally of the distal end of probe (100). In some examples, tissue sample window (139) exposes a gate assembly (not shown), such that the gate assembly is visible to an operator though probe (100). The gate assembly is generally configured to selectively arrest movement of the severed tissue sample within the fluid conduit between the cutter and the tissue sample holder (150). The gate assembly is enables the operator to temporarily cease progression of tissue samples for visual inspection though a sample window (139) of probe (100). At least a portion of the gate assembly is coupled to cutter to communicate rotational and translational motion of gate assembly to cutter. Thus, it should be understood that rotation and translation of cutter drive member (not shown) results in corresponding rotation and translation of cutter via the coupling between at least a portion of the gate portion and at least a portion of the gate assembly. In some examples, the gate assembly may be constructed in accordance with the teachings of U.S. application No. [Atty. Docket No. LEI 20011-SO-US.0642281], entitled “Apparatus to Allow Biopsy Sample Visualization During Tissue Removal,” filed on an even date herewith, the disclosure of which is incorporated by reference herein. Alternatively, probe (100) may simply lack a gate assembly, such that severed tissue samples are allowed to travel freely to tissue sample holder (150).

As described above, holster (200) of the present example is configured as a reusable portion, while probe (100) is configured as a disposable portion. In some contexts, it may be desirable to maintain sterility of reusable components during a biopsy procedure. Accordingly, in some instances it may be desirable to use holster (200) in connection with certain shielding features to maintain the sterility of holster (200), while also maintaining functionality of holster (200). Although some suitable features for maintaining the sterility of holster (200) are described below, it should be understood that other alternative configurations may be used in connection with biopsy device (10) without departing from the teachings herein.

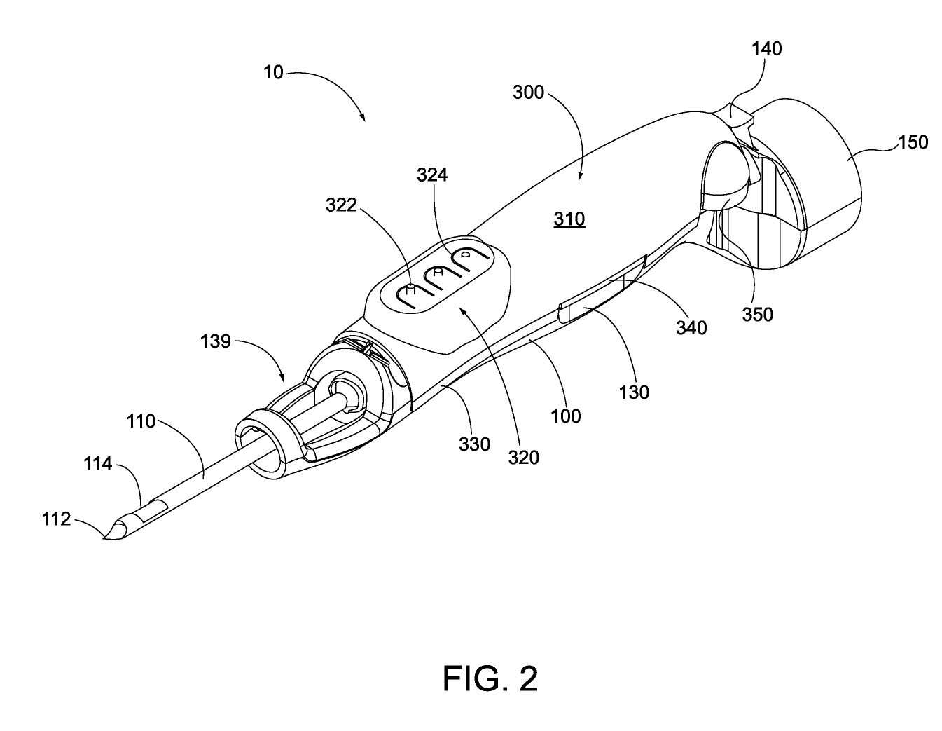

As shown in FIG. 2, a removable functional cover (300) of the present example is selectively attachable to holster (200) and is generally configured to cover a portion of holster (200) while leaving a substantial portion of probe (100) uncovered. In some examples, the term substantial portion referred to herein can include about 50% of the outer surface of probe (100) being uncovered. The typical functionality of removable functional cover (300) is to provide sterility protection of the various components of holster (200) and the outer housing (202) of holster (200). In the present configuration, holster (200) is a reusable component, while removable functional cover (300) is disposable. It should be understood that the use of the term “cover” herein should not be read as only requiring removable functional cover (300) to be placed on top of outer housing (210) of holster (200). For instance, as will be discussed in greater detail below, in the present example removable functional cover (300) includes a set of fasteners (340, 342) that engage holster (200) to secure removable functional cover (300) to biopsy device (10). Removable functional cover (300) includes housing (310), which encases outer housing (202) of holster (200) and the various components of holster (200).

As best seen in FIG. 3, housing (310) includes a distal opening (312) at the distal end of removable functional cover (300) and a proximal opening (314) on the proximal opening of removable functional cover (300). Removable functional cover (300) further includes a button actuation feature (320) integral to housing (310) and adjacent to the distal opening (312) of removable functional cover (300). Button actuation feature (320) of removable functional cover (300) is positioned to cover corresponding button panel (220) of holster (200) when removable functional cover (300) is attached to holster (200). Although not shown, it should be understood that button actuation feature (320) may also be positioned at other various portions of housing (310) of removable functional cover (300) to correspond to the location of button panel (220) of holster (200). Various other suitable configurations and operabilities of button actuation feature (320) will be apparent to those of ordinary skill in the art in view of the teachings herein.

Button actuation feature (320) comprises at least one protruding portion (322, 323) that corresponds to individual buttons (222) of the button panel (220) of holster (200). External protruding portion (322) may extend outwardly from button actuation feature (320) at varying lengths. As best seen in FIG. 2, each external protruding portion (322) of button actuation feature (320) may extend at a different length than an immediately adjacent external protruding portion (322). As will be understood, such varying lengths of external protruding portions (322) may provide some tactile feedback to an operator to promote differentiation among each button actuation feature (320).

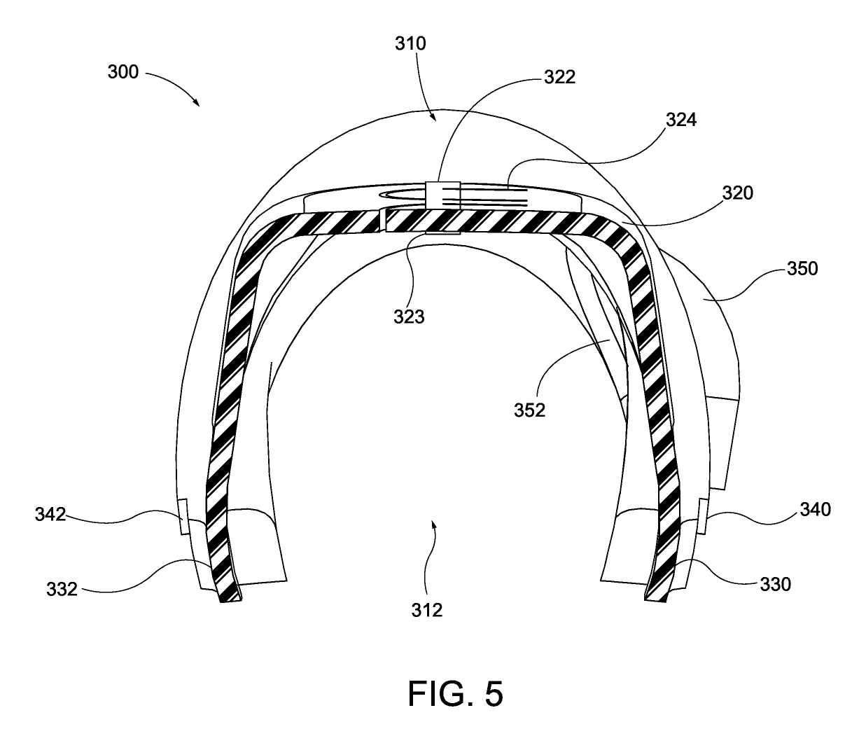

As best seen in FIG. 5, internal protruding portion (323) of button actuation feature (320) extends inwardly from button actuation feature (320) at varying lengths. The inward extension of internal protruding portion (323) allows for enhanced actuation of buttons (222) of holster (200) via button actuation feature (320) of removable functional cover (300). Protruding portions (322, 323) may be made from various materials that provide flexible movement upon manipulation (including plastic, elastic, elastomaterial, etc.). Other various materials for protruding portions (322, 323) may be provided as will be apparent to those of ordinary skill in the art in view of the teachings herein.

In the present example, button actuation feature (320) also includes aperture portion (324) for each protruding portion (322, 323). Although three external protruding portions (322) and aperture portions (324) are shown, it should be understood that button actuation feature (320) may include more or fewer protruding portions (322, 323) and/or aperture portions (324) to correspond to the number of buttons (222) on button panel (220) of holster (200). Aperture portion (324) comprises an elongated slot substantially surrounding protruding portions (322, 323). This elongated slot of each aperture portion (324) permits each button actuation feature (320) to be integral with housing (310), yet still movable relative to housing (310). This configuration additionally provides at least some resiliency to each button actuation feature (320) such that each button actuation feature (320) is resiliently biased in a non-actuated state away from buttons (222) of holster (200). Although each aperture portion (324) of the present example is shown as being generally uniform shape, size and length from button actuation feature (320) to button actuation feature (320), it should be understood that in other examples each aperture portion (324) may comprise varying shapes, sizes, and/or lengths. In addition, or in the alternative, each aperture portion (324) may be omitted entirely in some examples and instead each button actuation feature (320) can be replaced with a discrete material that provides flexible movement upon manipulation (including plastic, elastic, elastomaterial, etc.). In still other examples, aperture portion (324) can be used, but the elongated slot can be filled or covered with a thin membrane or other material to prevent fluids from penetrating functional cover (300) at aperture portion (324). Other various materials for aperture portion (324) may be provided as will be apparent to those of ordinary skill in the art in view of the teachings herein.

Returning to FIG. 3, housing (310) of removable functional cover (300) further includes sidewalls (330, 332) extending along the longitudinal length of housing (310) commencing at distal opening (312) and terminating at proximal opening (314). Sidewalls (330, 332) of removable functional cover (300) each also include at least one fastener (340, 342). Fasteners (340, 342) are positioned along sidewalls (330, 332) of housing (310) and attach to rim (230) of holster (200). Fasteners (340, 342) are positioned along sidewalls (330, 332) to correspond with the location of recesses (130, 132) of probe (100). Although not shown, it should be understood that fasteners (340, 342) may also be positioned at other various portions of sidewalls (330, 332) to correspond to the location of recesses (130, 132) of probe (100). Additionally, although not shown, it should be understood that more than one fastener (340, 342) may be included on sidewalls (330, 332) of housing (310). In some other versions, fasteners (340, 342) may employ other suitable fastening configurations to securely attach removable functional cover (300) to holster (200). While only a few exemplary fastener configurations have been described, other various configurations may be provided as will be apparent to those of ordinary skill in the art in view of the teachings herein.

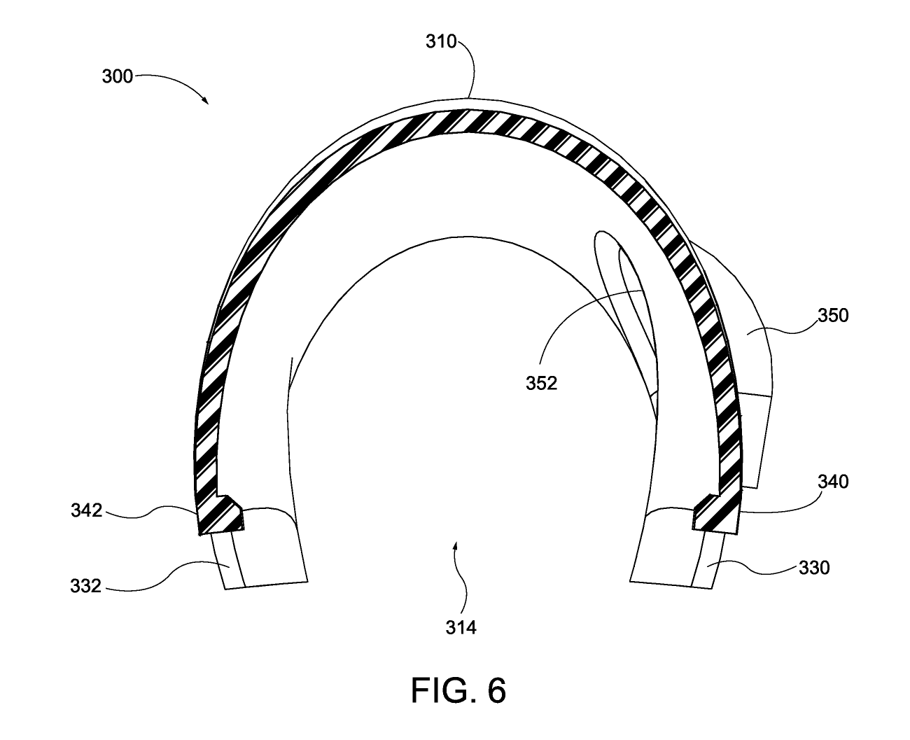

As best seen in FIG. 6, fasteners (340, 342) include inwardly extending portions that allow fasteners (340, 342) to securely attach to rim (230) of holster (200) when removable functional cover (300) is positioned on outer housing (202) of holster (200). In the present example, fasteners (340, 342) include quadrangular portions containing chamfered edges. Although not shown, it should be understood that fasteners (340, 342) may comprise various edge configurations or shapes for the inwardly extending portion (e.g. rounded, squared, rectangular, quadrilateral, etc.) or omit an extending portion on fastener (340, 342) altogether.

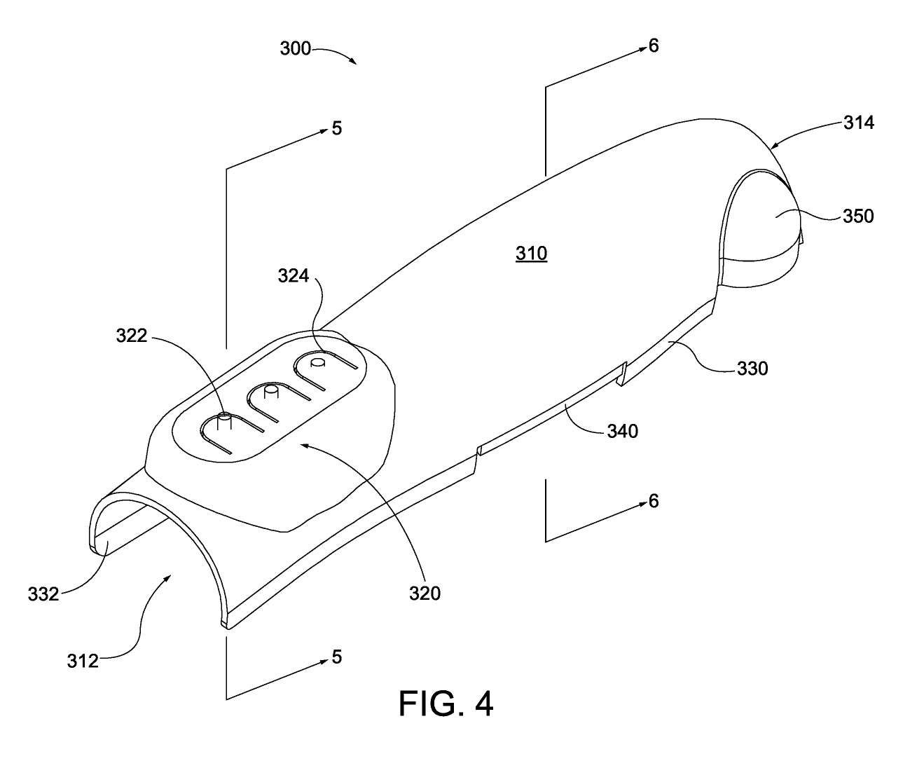

FIG. 4 shows fastener (340) along sidewall (330) at a middle portion of removable functional cover (300). Although sidewalls (330, 332) extend along housing (310) at a length greater than fasteners (340, 342) in the present example, it should be understood that fasteners (340, 342) may comprise various lengths along housing (310). As best seen in FIG. 6, in the present example sidewalls (330, 332) extend downwardly from housing (310) at a length greater than fasteners (340, 342). Although not shown, fasteners (340, 342) may extend downwardly from housing (310) at a length greater than sidewalls (330, 332).

FIG. 4 also shows removable functional cover (300) including chamber cover (350) integrally fixed to housing (310) at the proximal end of removable functional cover (300), adjacent to proximal opening (314). As best seen in FIGS. 5-6, chamber cover (350) extends outwardly from housing (310) and sidewall (330) to encase chamber (250) of holster (200). Channel (250) of holster (200) serves to provide a conduit for a cable (not shown) that connects holster (200) to a vacuum control module (not shown). In some other versions, chamber cover (350) may extend outwardly from sidewall (332) to correspond with the location of chamber (250) of holster (200). Chamber cover (350) includes an internal chamber opening (352) on the interior of housing (310) of removable functional cover (300) to accommodate the configuration and size of chamber (250) of holster (200). Although not shown, in some versions chamber cover (350) may not extend outwardly beyond housing (310) and sidewalls (330, 332). Other suitable components, configurations, and relationships will be apparent to those of ordinary skill in the art in view of the teachings herein.

An exemplary use of removable functional cover (300) is shown in FIGS. 2, 3, and 7. As shown in FIG. 3, the procedure begins with probe (100), holster (200), and removable functional cover (300) separate from one another. Outer housing (102) of probe (100) is sized and configured to receive holster (200) when an operator aligns locating feature (210) of holster (200) with receiving feature (120) of probe (100). Once the distal ends of holster (200) and probe (100) are aligned, an operator may lower the proximal end of holster (200) downwardly onto outer housing (102) of probe (100) until catch (240) of holster (200) securely engages prong (142) of probe (100), thereby coupling holster (200) to probe (100).

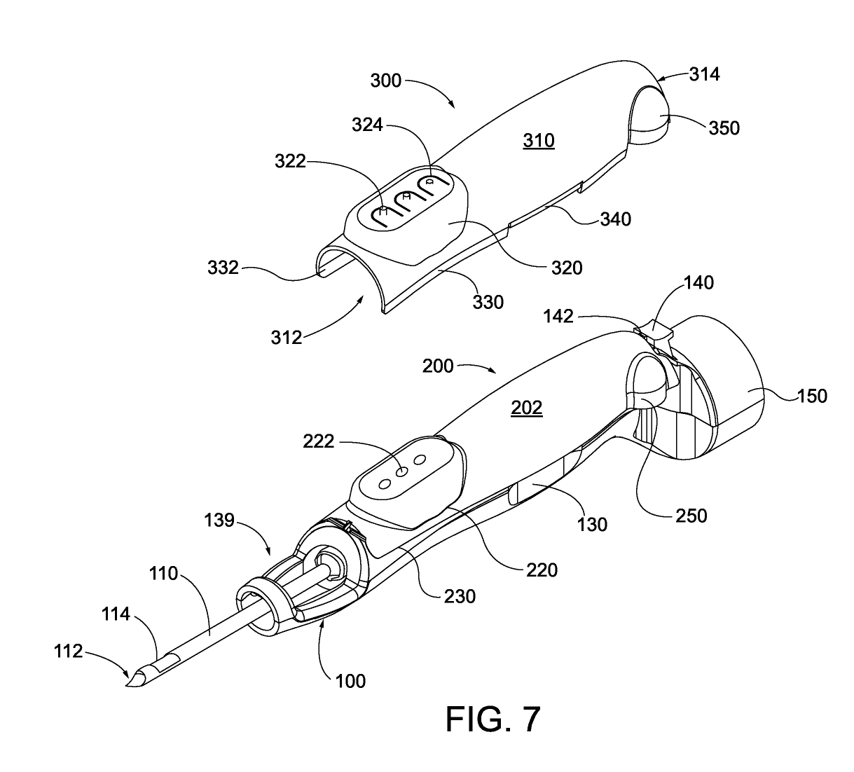

Once holster (200) is attached to probe (100) as shown in FIG. 7, removable functional cover (300) of the present example may be securely attached to holster (200) as shown in FIG. 1. Distal opening (312) of removable functional cover (300) is initially aligned with the distal end of holster (200) adjacent to locating feature (210). Proximal opening (314) of removable functional cover (300) is then aligned with the proximal end of holster (200) adjacent to catch (240). Upon an operator's application of downward force upon housing (310) of removable functional cover (300), fasteners (340, 342) securely engage rim (230) of holster (200) by means of a snap on configuration, thereby releasably securing removable functional cover (300) to holster (200). Housing (310) and sidewalls (330, 332) of removable functional cover (300) are sized and configured to securely fit outer housing (202) and rim (230) of holster (200), respectively. As described above, other various suitable type and quantities of fasteners (340, 342) may be provided on sidewalls (330, 332) of removable functional cover (300) to securely fasten removable functional cover (300) to holster (200) as will be apparent to those of ordinary skill in the art in view of the teachings herein. Housing (310) of removable functional cover (300) is configured to compress a seal when attached to the outer housing (202) of holster (200), thereby providing a sterile casing that prevents bodily fluids from infiltrating biopsy device (10).

Button panel (220) of holster (200) will be effectively encased by button actuation features (320) of removable functional cover (300) and chamber (250) of holster (200) will be effectively encased by chamber cover (350) of removable functional cover (300). Buttons (222) of button panel (220) are operable through button actuation feature (320) of removable functional cover (300) by the external protruding portions (322) and internal protruding portions (323) and aperture portions (324). The application of downward force by an operator onto the external protruding portion (322) effectively depresses the corresponding internal protruding portion (323) and aperture portion (324) toward button (222) of button panel (220), thereby allowing operation of the biopsy device (10).

Removable functional cover (300) serves as a functional cover to maintain sterility of holster (200) during a biopsy procedure or other similar procedure. Removable functional cover (300) may be detached from holster (200) by an operator and disposed of after a biopsy or other procedure has been completed. As seen in FIG. 7, Removable functional cover (300) and holster (200) will remain securely coupled until operator applies outward force upon fasteners (340, 342) of sidewall (330, 332) at recesses (130, 132) of probe (100). Recesses (130, 132) provide a depressed surface portion on probe (100) to allow an operator's hands to manually operate fasteners (340, 342). Upon an operator's application of force on fasteners (340, 342) of removable functional cover (300) at recesses (130, 132) of probe (100), fasteners (340, 342) extend outwardly from rim (230) of holster (200) and release rim (230) of holster (200) thereby decoupling the removable functional cover (300) and holster (200).

Once removable functional cover (300) is decoupled from holster (200), an operator may lift removable functional cover (300) off outer housing (202) of holster (200) and separate housing (310) of removable functional cover (300) from outer housing (202) of holster (200). Holster (200) and probe (100) will remain securely coupled until the operator applies downward force upon latch (140) of probe (100) which raises prong (142) away from catch (240) of holster (200) thereby decoupling the proximal ends of holster (200) and probe (100). At this point, the operator may lift holster (200) off outer housing (102) of probe (100) and separate locating feature (210) of holster (200) from receiving feature (120) of probe (100) to fully detach holster (200) from probe (100). Once removed, removable functional cover (300) may be disposed of by the operator while holster (200) may be subsequently reused in a following biopsy or other similar procedure due to its maintained sterility.

Other suitable configurations that may be employed for removable functional cover (300), in addition to providing sterility protection to holster (200), can be through noise reduction by including a protective coat of noise reducing material along the interior surface of removable functional cover (300). Another suitable configuration that may be used for removable functional cover (300) may include providing sterility protection to holster (200) in conjunction to an ergonomic improvement by adding a protective coat of a noise reducing material along the exterior surface of removable functional cover (300).

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A biopsy device comprising:
(a) a reusable body containing a drive mechanism;
(b) a disposable probe adapted to couple the drive mechanism of the reusable body in use, the disposable probe having a body and a needle extending distally from the body, a cutter associated with the needle and movably coupled to the drive mechanism, the reusable body having at least one button for operating the probe; and
(c) a sterile cover shaped and configured to couple to the reusable body, wherein the sterile cover includes a contact area for access to the at least one button.

2. The biopsy device of claim 1, wherein the sterile cover further includes a sterile membrane covering the contact area to prevent fluid flow through the contact area.

3. The biopsy device of claim 1, wherein the sterile cover further includes one or more fasteners configured to be locked to the reusable body in a snap fit configuration.

4. The biopsy device of claim 1, wherein the sterile cover includes a chamber cover, wherein the chamber cover is sized and configured to receive a chamber protrusion of the reusable body which received an electrical cable.

5. The biopsy device of claim 1, wherein the sterile cover is shaped to cover the reusable body and to leave a substantial portion of the body of the disposable probe uncovered.

6. The biopsy device of claim 1, further comprising a button actuator, wherein the button actuator is associated with the contact area of the sterile cover such that the button actuator is adapted to actuate the at least one button of the reusable body, wherein the button actuator is resiliently biased such that the button actuator is configured to return to an initial position after actuating the at least one button of the reusable body.

7. The biopsy device of claim 6, wherein the button actuator includes an external protruding portion configured to provide tactile feedback to an operator thereby indicating the location of the button actuator using tactile feedback.

8. The biopsy device of claim 6, wherein the button actuator includes an internal protruding portion configured to resiliently engage the at least one button of the reusable body.

9. The biopsy device of claim 6, wherein the button actuator is of integral construction with the sterile cover, wherein the button actuator and the sterile cover together define a gap between the sterile cover and the button actuator.

10. The biopsy device of claim 9, wherein the gap is filled or otherwise covered by a membrane, wherein the membrane is configured to prevent movement of fluid through the gap defined between the sterile cover and the button actuator.

11. The biopsy device of claim 9, wherein the sterile cover and the button actuator include a single homogeneous material.

12. The biopsy device of claim 1, wherein an interior surface of the sterile cover is coated with a noise reducing material.

13. The biopsy device of claim 1, wherein an exterior surface of the sterile cover is coated with a noise reducing material.

14. A biopsy device comprising:
(a) a reusable body including a driver and a button panel, the button panel including one or more buttons;
(b) a disposable probe including a needle, a cutter, and a cutter actuator, wherein the cutter actuator is configured to move the cutter relative to the needle to sever a tissue sample, wherein the disposable probe is configured to couple to the reusable body to thereby place the driver of the reusable body into communication with the cuter actuator; and
(c) a sterile cover shaped and configured to house an exterior surface of the reusable body therein when the disposable probe is coupled to the reusable body, wherein the sterile cover includes a button cover, wherein the button cover is configured to align with the button panel of the reusable body.

15. The biopsy device of claim 14, wherein the reusable body includes a proximal coupler and a distal coupler, wherein the proximal coupler and the distal coupler are configured to engage the disposable probe regardless of whether the sterile cover is coupled to the reusable body or not.

16. The biopsy device of claim 15, wherein the sterile cover is configured to maintain sterility of the reusable body while permitting engagement between the proximal coupler, the distal coupler and the disposable probe.

17. The biopsy device of claim 14, wherein the button cover of the sterile cover is includes an elastomer configured to prevent fluid flow through the button cover while permitting at least a portion of the button cover to resiliently flex.

18. The biopsy device of claim 14, wherein the reusable body defines a protrusion configured for egress of at least one electrical cable from the reusable body, wherein the sterile cover defines a chamber to encompass at least a portion of the protrusion.

19. A biopsy system, the biopsy system comprising:
(a) a biopsy device, the biopsy device including:
(i) a holster, the holster including a housing, a motor disposed within the housing, and a button disposed on an exterior of the housing, and
(ii) a probe including a handle, a needle extending distally from the handle, and a cutter movable relative to the needle, wherein the handle of the probe is configured to couple to at least a portion of the holster, wherein the motor of the holster is configured to move the cutter when the probe is coupled to the holster; and
(b) a sterile cover configured to encompass a portion of the holster exposed relative to the probe when the probe is coupled to the holster, wherein the sterile cover includes a sealed actuator, wherein the sealed actuator is configured to actuate the button of the holster.

20. The biopsy system of claim 19, wherein the handle of the probe defines a recess configured to receive at least a portion of the sterile cover therein to thereby permit coupling between the holster and the sterile cover.
